(19) United States
(12) Patent Application Publication
(10) Pub. No.: US 2010/0297228 A1
(43) Pub. Date: Nov. 25, 2010

(54) UNIVERSAL COATING FOR IMPRINTING IDENTIFICATION FEATURES

(75) Inventors: Cedric Loiret-Bernal, Evanston, IL (US); Nabil Amro, Chicago, IL (US); Sandeep Disawal, Chicago, IL (US); Bjoern Rosner, Chicago, IL (US); John E. Bussan, Naperville, IL (US); Bo He, Schaunburg, IL (US)

Correspondence Address: FOLEY AND LARDNER LLP;SUITE 500, 3000 K STREET NW, WASHINGTON, DC, 20007, US

(73) Assignee: Nanolnk, Inc.

(21) Appl. No.: 12/260,911

(22) Filed: Oct. 29, 2008

Related U.S. Application Data
(60) Provisional application No. 60/983,562, filed on Oct. 29, 2007.

(51) Int. Cl. A61K 9/44; B05D 3/12; A61P 43/00

(52) U.S. Cl. 424/467; 427/2.23

(57) ABSTRACT

To improve anticounterfeiting protection, a method for imprinting pharmaceutical unit compositions comprising: providing a pharmaceutical unit composition, partially coating the exterior of the composition with a coating, stamping the coating with a stamp comprising a plurality of identification features, wherein identification features from the stamp are at least partially transposed in the coating and form a barcode, wherein the plurality of identification features comprise at least one lateral dimension of about 1,000 nm or less. Other objects can be coated and stamped including currency and luxury goods.

### RELATED APPLICATION

This application claims priority to U.S. provisional patent application 60/983,562 filed Oct. 29, 2007 which is hereby incorporated by reference in its entirety.

### BACKGROUND

A global need exists to reduce counterfeiting, piracy, and other types of intellectual property and criminal theft. In addition, a global need exists to provide for tracking of objects of value through the chain of commerce and across borders and prevent product diversion. The integrity of global supply chains needs to be improved. Overt and covert solutions to the problem have been proposed, both at the unit level and the package level. These approaches include color manipulation, marking schemes, threads, RFID, taggants, color shifting dyes, holograms, and encrypted RFID. These needs are particularly acute in the pharmaceutical industry. See, for example, Wertheimer et al., “Counterfeit Pharmaceuticals: Current Status and Future Projections,” J. American Pharmacists Association, November/December 2003, vol. 43, no. 6, 710-718; Counterfeiting Exposed: Protecting Your Brand Name and Customers, D. M. Hopkins, L. T. Kontnik, M. T. Turnage (Wiley, Ed. 2003) including Chapters 4 and 12.

One approach is to provide packaging with treatments to prevent anti-counterfeiting. However, materials within the package can be removed from the packaging. Approaches are needed based on the unit article rather than the packaging alone.

One approach is to provide a genuine article or composition with identification features. Nanotechnology provides new avenues to create identification features which are difficult or impossible for counterfeiters to reproduce. In particular, bar code technology can be a powerful anti-counterfeiting method.

Literature references include:

U.S. Patent Publication 2006/0226234 describes oral solid dosages having overt printed or etched markings.

U.S. Pat. No. 7,083,805 describes high resolution microrelief hologram structures.

U.S. Patent Publication 2006/0087051 describes an edible dosage form having optical elements for identification.

U.S. Pat. No. 5,683,718 describes providing embossed tablets with enteric coatings for identification purposes.

U.S. Pat. No. 5,002,775 describes tablets having clear marks which are impressed thereon comprising different color tones and subsequently coated.

U.S. Patent Publication 2005/0180599 describes surface texturing to encode a plural-bit code.

U.S. Pat. No. 4,168,321 describes press-formed tablet with coloring agent.

U.S. Pat. No. 5,376,771 describes a laser drilling process for fabricating holes in pharmaceutical dosage forms.

U.S. Pat. No. 6,543,692 describes bar code schema for identification of solid form drugs.

U.S. Pat. No. 6,799,725 describes pill imprinting with microbarcode.

In particular, a need yet exists to improve the reproducibility and versatility of anticounterfeit markings from pill to pill, particularly for barcode technology, and to increase the levels of anticounterfeiting protection.

### SUMMARY

Various embodiments are described herein, including articles, instruments, compositions, methods of manufacturing, and methods of using.

One embodiment provides a method for imprinting pharmaceutical unit compositions, comprising: providing a pharmaceutical unit composition, partially coating the exterior of the composition with a coating, stamping the coating with a stamp comprising a plurality of identification features, wherein identification features from the stamp are at least partially transposed in the coating to form a barcode, wherein the plurality of identification features comprise at least one lateral dimension of about 1,000 nm or less. In another embodiment, the plurality of identification features can comprise at least one lateral dimension of about 10,000 nm or less, or can comprise at least one lateral dimension of about 5,000 nm or less, or for example, about 100 nm to about 5 microns, or about 100 nm to about 1 micron. Another embodiment is the pharmaceutical composition prepared by this method.

Another embodiment provides a method for imprinting objects comprising: providing an object, partially coating the exterior of the object, stamping the coating with a stamp comprising a plurality of identification features, wherein identification features from the stamp are at least partially transposed in the coating to form a barcode, wherein the plurality of identification features comprise at least one lateral dimension of about 1,000 nm or less. Another embodiment is the object prepared by this method.

Another embodiment comprises a method for imprinting pharmaceutical unit compositions comprising: providing a pharmaceutical unit composition, partially coating the exterior of the composition with a coating which is adapted to receive a barcode stamp and also bind to the pharmaceutical unit composition, stamping the coating with a stamp comprising a plurality of identification features, wherein temperature, pressure, and time are adapted so that identification features from the stamp are at least partially transposed in the coating and form a barcode in the coating, wherein the plurality of identification features comprise at least one lateral dimension of about 1,000 nm or less.

Another embodiment is a method for imprinting pharmaceutical unit compositions comprising: providing a pharmaceutical unit composition, partially coating the exterior of the composition with a coating, wherein the coating comprises at least one fluorescent dye, stamping the coating with a stamp comprising a plurality of identification features, wherein identification features from the stamp are at least partially transposed in the coating, wherein the plurality of identification features comprise at least one lateral dimension of about 1,000 nm or less.

Also provided is a method for imprinting objects comprising: providing an object, partially coating the exterior of the object, wherein the coating comprises at least one fluorescent dye, stamping the coating with a stamp comprising a plurality of identification features, wherein identification features from the stamp are at least partially transposed in the coating, wherein the plurality of identification features comprise at least one lateral dimension of about 1,000 nm or less.

Also provided is an imprinted pharmaceutical unit composition comprising: pharmaceutical unit composition, a coating disposed on the exterior of the composition, a plurality of identification features in the coating and forming a barcode in the coating, wherein the plurality of identification features comprise at least one lateral dimension of about 1,000 nm or less.

Also provided is an imprinted object comprising: an object, a coating disposed on the exterior of the object, a plurality of identification features in the coating and forming a barcode in the coating, wherein the plurality of identification features comprise at least one lateral dimension of about 1,000 nm or less.

Another embodiment is an imprinted object comprising: an object, a coating disposed on the exterior of the object, the coating comprising at least one fluorescent dye, a plurality of identification features in the coating and forming a barcode in the coating, wherein the plurality of identification features comprise at least one lateral dimension of about 1,000 nm or less.

Another embodiment provides a method for imprinting pharmaceutical unit compositions, comprising: providing a pharmaceutical unit composition, partially coating the exterior of the composition with a coating, stamping the coating with a stamp comprising a plurality of identification features, wherein identification features from the stamp are at least partially transposed in the coating to form a barcode, wherein the plurality of identification features comprise at least one lateral dimension of at least about 1,000 nm. In an embodiment, the upper lateral dimension can be about 10 microns.

Another embodiment is a method for imprinting objects comprising: providing an object, partially coating the exterior of the object, stamping the coating with a stamp comprising a plurality of identification features, wherein identification features from the stamp are at least partially transposed in the coating to form a barcode, wherein the plurality of identification features comprise at least one lateral dimension of at least about 1,000 nm. In an embodiment, the upper lateral dimension can be about 10 microns.

Another embodiment provides a method for imprinting pharmaceutical unit compositions comprising: providing a pharmaceutical unit composition, partially coating the exterior of the composition with a coating which is adapted to receive a barcode stamp and also bind to the pharmaceutical unit composition, stamping the coating with a stamp comprising a plurality of identification features, wherein temperature, pressure, and time are adapted so that identification features from the stamp are at least partially transposed in the coating and form a barcode in the coating, wherein the plurality of identification features comprise at least one lateral dimension of at least about 1,000 nm. In an embodiment, the upper lateral dimension can be about 10 microns.

Another embodiment provides a method for imprinting pharmaceutical unit compositions comprising: providing a pharmaceutical unit composition, partially coating the exterior of the composition with a coating, wherein the coating comprises at least one fluorescent dye, stamping the coating with a stamp comprising a plurality of identification features, wherein identification features from the stamp are at least partially transposed in the coating, wherein the plurality of identification features comprise at least one lateral dimension of at least about 1,000 nm. In an embodiment, the upper lateral dimension can be about 10 microns.

Another embodiment provides a method for imprinting objects comprising: providing an object, partially coating the exterior of the object, wherein the coating comprises at least one fluorescent dye, stamping the coating with a stamp comprising a plurality of identification features, wherein identification features from the stamp are at least partially transposed in the coating, wherein the plurality of identification features comprise at least one lateral dimension of at least about 1,000 nm. In an embodiment, the upper lateral dimension can be about 10 microns.

Another embodiment provides an imprinted pharmaceutical unit composition comprising: pharmaceutical unit composition, a coating disposed on the exterior of the composition, a plurality of identification features in the coating and forming a barcode in the coating, wherein the plurality of identification features comprise at least one lateral dimension of at least about 1,000 nm. In an embodiment, the upper lateral dimension can be about 10 microns.

Another embodiment provides an imprinted object comprising: an object, a coating disposed on the exterior of the object, the coating comprising at least one fluorescent dye, a plurality of identification features in the coating and forming a barcode in the coating, wherein the plurality of identification features comprise at least one lateral dimension of at least about 1,000 nm. In an embodiment, the upper lateral dimension can be about 10 microns.

Alternatively, provided herein is an imprinted object comprising: an object, a coating disposed on the exterior of the object, a plurality of identification features in the coating and forming a barcode in the coating, wherein the plurality of identification features comprise at least one lateral dimension of at least about 1,000 nm. In an embodiment, the upper lateral dimension can be about 10 microns.

Advantages of at least one embodiment include among others improved reproducibility and versatility and/or less optimization as the printing process is done on different pills or objects. For example, one can have large batch to batch variability even in the same manufactured product, and the present methods can help reduce this variability. In addition, the coating provides one or more additional layers of protection, supplementing the identification features printed on the pills or objects.

### BRIEF DESCRIPTION OF THE DRAWINGS

FIG. 1 illustrates a schematic diagram of an embodiment.

FIG. 2 provides an SEM image of a wax patch on a pharmaceutical tablet.

FIG. 3 provides an SEM image of an imprint on the wax patch of FIG. 2.

FIG. 4 provides an optical image of a logo on a wax patch on a pharmaceutical tablet.

FIG. 5 provides an SEM image of a logo on a wax patch on a pharmaceutical tablet.

FIG. 6 provides SEM images of nanofeatures and barcodes created on a wax patch.

FIG. 7 provides optical images for HPMC patch on a pharmaceutical tablet.

FIG. 8 provides SEM images for HPMC patch on a pharmaceutical tablet.

FIG. 9 provides more optical images for HPMC patch on a pharmaceutical tablet.

FIG. 10 provides more SEM images for HPMC patch on a pharmaceutical tablet.

FIG. 11 provides optical images for an epoxy patch on different surfaces.

FIG. 12 provides SEM images for an epoxy patch on a commercial pharmaceutical box.

FIG. 13 provides SEM images for an epoxy patch on a commercial pharmaceutical box.

FIG. 14 provides SEM images for an epoxy patch on an aluminum holder.

FIG. 15 illustrates a fluorescent image of a fluorescent embodiment for patch on tablet.

FIG. 16 illustrates another fluorescent image of a fluorescent embodiment with logo.

FIG. 17 illustrates another fluorescent image of a fluorescent embodiment with logo, higher magnification.

FIG. 18 illustrates a masking of the fluorescent layer.

FIG. 19 illustrates an optical image of masking of the fluorescent layer.

FIG. 20 illustrates an optical image of masking of the fluorescent layer.

FIGS. 21A-21B provide SEM images of a PDMS patch and a Nanoencryption™ mark on the patch, respectively.

FIGS. 22A-22C provide SEM images of micro- and nano-scale features on PDMS patch on a pharmaceutical 1 mg tablet.

### DETAILED DESCRIPTION

### Introduction

All references cited hereinafter are incorporated by reference in their entirety.

Priority U.S. provisional patent application 60/983,562 filed Oct. 29, 2007 is hereby incorporated by reference in its entirety including figures, claims, and various embodiments.

One embodiment provides a method for imprinting objects comprising: providing an object, partially coating the exterior of the object, stamping the coating with a stamp comprising a plurality of identification features, wherein identification features from the stamp are at least partially transposed in the coating and form a barcode, wherein the plurality of identification features comprise at least one lateral dimension of about 1,000 nm or less.

One particularly preferred embodiment provides a method for imprinting pharmaceutical unit compositions comprising: providing a pharmaceutical unit composition, partially coating the exterior of the composition with a coating, stamping the coating with a stamp comprising a plurality of identification features, wherein identification features from the stamp are at least partially transposed in the coating and form a barcode, wherein the plurality of identification features comprise at least one lateral dimension of about 1,000 nm or less.

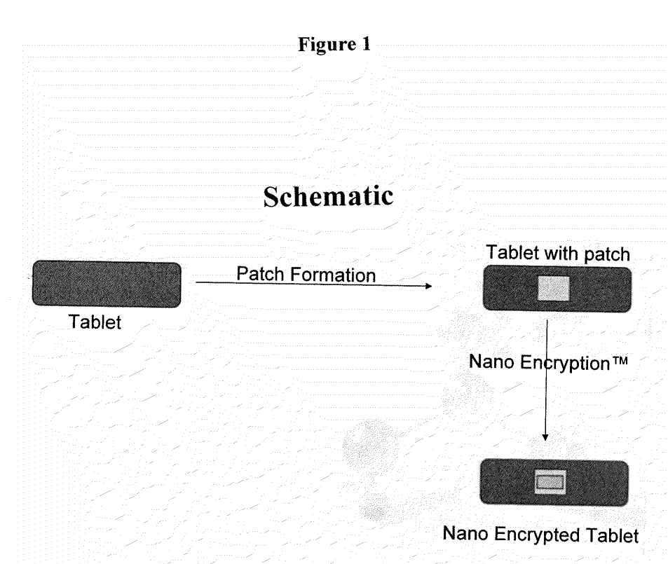

FIG. 1 illustrates an embodiment, wherein a tablet is subjected to a coating or patch formation process to form a tablet with a patch. Then, the patch is subjected to formation of identification features such as those needed to form a barcode.

Various background embodiments which can be used for practicing the presently claimed inventions are described in for example U.S. patent application Ser. No. 11/109,877 filed Apr. 20, 2005 to Loiret-Bernal et al. including objects, pharmaceutical compositions, identification regions and features, stamps, stamping methods, instruments, and detection methods. Additional embodiments, particularly for instruments, are described in U.S. patent application Ser. No. 11/305,327 to Loiret-Bernal et al. filed Dec. 19, 2005 and for stamps Ser. No. 11/305,326 to Loiret-Bernal et al. filed Dec. 19, 2005. Covert and overt stamp technology is described in Ser. No. 11/305,189 filed Dec. 19, 2005. Methods for reading stamped objects can be found in Ser. No. 11/519,199 filed Sep. 12, 2006 to Loiret-Bernal et al.

For commercial production, continuous rather than batch methods of production can be used such as continuous delivery of objects and compositions to a zone for coating and stamping, following by continuous removal of coated and stamped objects and compositions from the zone.

U.S. Pat. No. 7,083,805 for example describes methods of forming microrelief surfaces on pharmaceutical substances.

### Objects

A wide variety of objects can be subjected to the coating and stamping methods described herein. For example, pharmaceutical compositions, currency, watches, medicines, perfumes, aircraft and automobile parts including spare parts, toys, music, video, and software. Other examples include medical devices, medical stents, batteries, electronic components and any high value items. Still other examples include stock certificates, identification cards, drivers license, artwork, hand-crafted instruments, guitars, violins, vials, and the like. The objects can present surfaces comprising a wide variety of materials including polymers, metals (including aluminum), ceramics, glass, paper, cardboard, composites, tablets, packaging, tablet packaging, and the like. The surface can be relative smooth or rough, flat or curved, and the like.

The object can be a smooth object or a rough object. The object can be compressed granules as in for example a tablet. In one embodiment, the surface can be made relatively smooth by application of the coating before imprinting or stamping.

Particularly important objects are compositions and pharmaceutical compositions. The problems of pharmaceutical and drug compositions which can be subjected to counterfeiting are described in, for example, the FDA report “Combating Counterfeit Drugs” February 2004 and “FDA's Counterfeit Drug Task Force Interim Report” October 2003 and other technical literature provided in this patent application. A wide variety of pharmaceutical drugs and compositions, and there function, are known in the art and are generally described in, for example, (i) Physicians' Desk Reference, 49 Ed, 1995 including brand name and generic drugs, and (ii) Goodman and Gilman's, The Pharmacological Basis of Therapeutics, 2001. Pharmaceutical compositions can be useful for both human and animal treatment.

### Pharmaceutical Unit Composition

The pharmaceutical composition is not particularly limited and a wide variety of pharmaceutical compositions are known in the art including pharmaceutical drugs in various shapes and sizes such as pills, tablets, caplets, capsules, and the like, and vials. Pill embossing and its variants can be the subject of the invention for pharmaceutical compositions. For example, tablets and caplets can be used after compressing or coating. Two-piece hard gelatin can be used, before or after filling with powder, gel, or liquid. Pill surface structures can include sugar shell, soft-shell, dipped or enrobed, enteric, or aqueous coated tablets, waxed tablets, and dry coatings.

The pharmaceutical compositions can contain active ingredients and passive ingredients, and in different embodiments, these can be distributed differently. The pharmaceutical composition can have an exterior region or surface which can be processed to include one or more identification regions by, for example, imprinting or embossing, whether hot or cold embossing. Hence, the pharmaceutical composition can be processible and susceptible to, for example, heat and pressure effects which allow for imprinting or embossing. The pharmaceutical composition generally can comprise an active pharmaceutical ingredient (API) but the invention is not particularly limited to how the API is distributed throughout the pharmaceutical composition. For example, the API could be in the interior or could be subjected to a coating process. A composition could be provided with the identification feature and then combined with the API. Hence, for example, the surface of the pharmaceutical composition may have little if any of the API but yet it is still part of the pharmaceutical composition. The API can be a solid, liquid, or gel API as long as the ultimate pharmaceutical composition can be processed to include the identification features described herein.

Examples of active ingredients include pharmaceuticals, minerals, vitamins, nutraceuticals, oral care agents, flavorants, and mixtures thereof. Examples of pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, osteoporosis preparations, respiratory agents, sleepaids, urinary track agents, and mixtures thereof. See for example US Patent Publication 2006/0088586 for pharmaceutical technology, ingredients therein generally, and specific examples.

The surface of the pharmaceutical composition can be an exterior surface which represents an interface with air. In addition, however, the surface of the pharmaceutical composition could be an interior surface. For example, an interior surface can be prepared by generating a desired surface having desired surface features and then combining that surface with another composition so that the desired features are no longer directly exposed to the air but can be detected, even though they are now interior surfaces. For example, an identification region can be generated and then overcoated with a protective film, coating, or layer, which include but are not limited to thin conformal films. The surface of the pharmaceutical composition can be generally flat and smooth, although at the scale of the identification features described herein the surface can be generally rougher. Or the surface can be non-flat or curved, including spherical, oval, or bi-convex. An interior surface can be desirable to avoid scratching or rubbing of the identification region. Alternatively, the identification region may comprise one or more features, which protect the information-bearing part from erasure or damage. For example, a raised ring or frame surrounding the identification features may avoid mechanical abrasion of the identification.

A variety of pharmaceutical shapes are described in for example US Patent Publication 2006/0088586 paragraphs 141-181.

Uncoated tablets can be used.

Coating Composition

A coating composition can be applied to the pharmaceutical unit composition. In many cases, the pharmaceutical unit composition can be approved by regulatory authorities as is but is further modified by the coating composition and stamping to facilitate anticounterfeiting protection.

In most embodiments, the coating material is selected because it has at least some of the following characteristics:

(1) Have suitable glass transition temperature, T_{g} (for example, for polymers), since this parameter will impact the ability to apply the patch and to imprint on it. For example, glass transition temperature can be less than 25° C. or greater than 25° C. The glass transition temperature can be, for example, from −150° C. to about 300° C.

(2) Be easy to modify chemically in order to improve adhesion to the tablet surface.

(3) Have reasonable cost at required purity.

(4) Have manageable die stamp fouling tendency during imprinting.

(5) Be chemically stable during imprinting process.

(6) Be thermally stable during imprinting process.

(7) Have adequate shelf-life after imprinting, as demonstrated by stability testing under various environmental conditions.

(8) Have acceptable curing time after deposition on tablet.

(9) Have acceptable curing temperature after deposition on tablet.

(10) Have suitable viscosity (affects flow properties during deposition on tablet surface).

(11) Have excellent adhesion on tablet surface.

(12) Have excellent abrasion resistance after deposition and imprinting.

(13) Have suitable color (clear preferred).

(14) Be compatible with solvents other than water that are acceptable for pharmaceutical tablet manufacture; good solubility and safety.

In one embodiment, the coating composition can comprise a composition which is generally recognized as safe by the US Food and Drug Administration (GRAS). In many cases, they will be described in GRAS notices. These include, for example, soy isoflavone extract, solin oil, sodium bisulfate, transglutaminase from Streptoverticillium mobaraense, tasteless smoke, and the like.

The coating can comprise at least one thermoplastic or thermomoldable or thermoformable material. The coating can comprise a synthetic organic polymer. The coating can comprise at least one soluble material, including for example a water soluble material, water swellable material, or a material soluble in organic solvents.

In one embodiment, the coating can comprise for example gelatin, modified cellulose, modified food starch, wax or waxes, vegetable gums, and combinations thereof. Other examples of polymers for coatings can be found in for example US Patent Publication 2006/0088586 at paragraphs 129-132.

In one embodiment, the coating comprises a cellulose polymer including a soluble cellulose polymer. It can be a modified cellulose material. The polymer can comprise a cellulose backbone and substituents on the side groups which provide solubility such as for example alkyl. Side groups can be ionic or neutral. For example, the polymer can be hydroxypropylcellulose (HPC) or hydroxypropylmethylcellulose (HPMC). The molecular weight of the cellulose can be adapted for a particular application. It is not particularly limited but can be for example about 5,000 to about 1,000,000. Mixtures and combinations can be used.

In one embodiment, the coating can comprise a polymer including for example a synthetic polymer such as for example epoxy (e.g., Epoxy 377 Epotek).

In one embodiment, a polymer such as an elastomer can be used. For example, silicone elastomer such as, for example, poly(dimethylsiloxane) (PDMS) can be used as the patch coating material.

In one particularly preferred embodiment, the coating comprises wax. Examples of waxes include carnauba, paraffin, candelia, and any wax extract. One can select a wax with a desired melting point. Mixtures can be used.

The coating composition can be adapted to provide excellent adhesion to the object to be stamped such as for example a pharmaceutical unit composition, as well as the ability to be stamped.

If desired, the coating can comprise one or more additives such as plasticizer or colorant or surfactant or adherent or oil.

Other coating materials can include cellulose derivatives, carboxymethylcellulose, carboxymethyl hydroxyethylcellulose, cellulose acetate, diethylaminoethyl cellulose, methyl cellulose, methyl ethyl cellulose, microcrystalline cellulose, cellulose triacetate, ethyl cellulose, hydroxypropyl cellulose (HPC), hydroxypropyl methyl cellulose (HPMC), zein, purified shellac, collagen, gelatin, hydroxylated lecithin, ethylene oxide/propylene oxide co-polymer, methyl methacrylate, polyethylene, oxidized polyethylene, polyisobutylene, cross-linked polystyrene. Mixtures can be used.

The coating can comprise one or more of the materials described above.

### Coating Method

Various embodiments can be used to apply the coating to the exterior of the unit pharmaceutical composition so as to at least partially coat the exterior. In general, one coating patch can be applied. Solution based methods can be used. Solvent systems can be aqueous or organic. In the alternative, melt based methods can be used without solvent.

Methods include, for example, microdispensing or nanodispensing including, for example, ink jet printing, electrospray, spray coating, dip coating, injection molding, pin transfer or spotting, pad transfer, thermal ribbon transfer, micropipetting, nanopipetting and roller band. See, for example, US Patent Publication 2006/0088586 for coating methods at paragraph 135 and references cited therein. Delivery of patch might involve heat prior to deposition or heat after deposition to provide curing. Any curing method common in the polymer industry such as UV, IR, and laser can be used.

The thickness of the coating can be for example from about 10 nm up to about 100 microns or even greater than 100 microns. For example, thickness can be about 500 nm to about 100 microns, or about one micron to about 50 microns. The coating can be thick enough so that stamping only indents the coating and not the material under the coating. For example, stamping may make an imprint about 7 microns to about 35 microns, wherein a coating about 40 microns or more, or about 50 microns or more can be used.

The coating can also be characterized by a volume. Examples include about 0.1 nL to about 100 nL, or about 1 nL to about 75 nL. An example is 25 nL. Suitable thickness can depend on for example the dispensing method and patch material.

If desired, multiple coating layers can be applied.

Coating can be cured by for example heating before further processing.

### Stamps, Stamp Methods, and Conditions

Stamping can be carried out using stamps and instruments as described in the US patent applications cited in the introduction section, including U.S. patent application Ser. No. 11/109,877 filed Apr. 20, 2005 to Loiret-Bernal et al.; U.S. patent application Ser. No. 11/305,327 to Loiret-Bernal et al. filed Dec. 19, 2005; and U.S. patent Ser. No. 11/305,326 to Loiret-Bernal et al. filed Dec. 19, 2005.

Stamping can be carried out so that only the coating is imprinted without any imprinting of the underlying object or pharmaceutical composition. Alternatively, stamping can be carried out so that imprinting passes through the coating into the underlying object or pharmaceutical composition.

The stamped pharmaceutical compositions, as well as other objects and compositions, can be made with one or more stamps which provide the surface with the identification feature. The invention provides a stamp for use in making a pharmaceutical composition, or other objects and compositions, the stamp comprising a surface having at least one identification region, and the region having at least one identification feature. In general, the stamps can be master stamps and can be used repeatedly, or can be used to produce other stamps.

The stamp size can be for example about 3 square mm or less, or about 2 square mm or less, or about 1 square mm or less, or about 0.6 square mm or less, or about 0.2 square mm to about 5 square mm.

Because the stamp can have a shape which is transferred to the pharmaceutical composition, or other objects and compositions, the dimensions described herein for the identification features and identification regions can also be used to describe the stamp. For example, a stamp which has an identification feature having a 100 nm height can result in a pharmaceutical composition, or other composition or object, having an identification feature with 100 nm height. The stamp's 100 nm positive protrusion can produce a 100 nm negative inversion.

In particular, the identification regions and features on the stamp can be characterized by dimensional measurements such as lateral dimensions or vertical dimensions with respect to the surface. Conventional methods can be used to measure these dimensions including methods described herein and the working examples. Conventional data processing including image processing, pattern recognition, curve fitting and optical character recognition (OCR) can be carried out to provide dimensions and average dimensions and generally to provide useful data.

The identification regions of the stamp can each have one or more identification features which can be characterized by a lateral dimension with respect to the surface. The lateral dimension can be, for example, a width or a length such as, for example, a circle diameter or a line width, or the relative or absolute position compared to a known mark. The lateral dimension is different from a vertical dimension such as height. For an identification feature which is a line, the lateral dimension of length can be sufficiently long that it can be viewed with the naked eye or an optical microscope, whereas the lateral dimension which is width can be sufficiently small that it cannot be seen with a naked eye or optical microscope. The size of the lateral dimensions can be sufficiently small so that the identification features are invisible to the naked eye and difficult to detect by conventional, simple methods. Rather, difficult, relatively expensive methods can be used to detect small identification features including microscopic and nanoscopic features. At least one of the lateral dimensions can be made small. For example, the identification feature of the stamp can have a lateral dimension of, for example, about 500 microns or less, or about 400 microns or less, or about 300 microns or less, or more particularly, about 250 microns or less, or more particularly, about 100 microns or less, or more particularly, about 10 microns or less, or five microns or less. Or the identification feature can have a lateral dimension of, for example, about one micron or less, or more particularly, about 500 nm or less, or more particularly, about 250 nm or less, or more particularly, about 100 nm or less. There is no particular limit to how small the lateral dimension can be as long as the identification feature can be detected. For example, the lateral dimension can be at least about 1 nm, or more particularly, at least about 10 nm, or more particularly, at least about 100 nm, or more particularly at least about one micron, such as at least 3 microns. Hence, exemplary ranges for the lateral dimension include about one nm to about 500 microns, about 10 nm to about 100 nm, about 100 nm to about one micron, and about one micron to about 500 microns.

For barcodes on the stamp, the line length is not particularly limited but can vary from nanoscopic to microscopic. For example, lines can be about one micron to about 50 microns long, or about 5 microns to about 25 microns long, and yet have a line width of only about 50 nm to about 150 nm wide.

The identification features on the stamp can be in the form of a pattern of repeating features such as dots or lines, wherein the features are characterized by an average lateral dimension such as average circle diameter or line width. The lateral size dimensions described herein can be computed into average lateral dimensions.

The identification features on the stamp can have a vertical dimension such as a height dimension or a depth dimension, and these terms are used interchangeably and for both positive structures and negative structures. The height dimension is not particularly limited and can be, for example, about one micron or less, or more particularly, about 500 nm or less, or more particularly about 250 nm or less, or more particularly about 150 nm or less. Alternatively, the dimension can be in the micron range, such as about 1 micron or more, such as 3 microns or more. There is no particular lower limit to the height dimension as long as the identification feature can be detected. The height dimension can be, for example, about one nm or more, or about 10 nm or more, or about 25 nm or more. Exemplary ranges can be, for example, about one nm to about one micron, or about 10 nm to about 500 nm, or about 25 nm to about 250 nm. Alternatively, the range can be about one micron or more, such as 1.5 microns or more, such as 3 microns or more. Again, if a pattern of repeating identification features are used, the vertical dimension can represent an average dimension.

In addition to the lateral dimension and the height dimension for identification features on the stamp, the invention can be also characterized by a separation dimension which represents the distance between the identification features such as a separation distance or a pitch. In other words, the one or more identification features can be separated from each other by a particular distance, and this distance can be an average distance for an array of identification features. For example, if the identification features are a series of lines, a distance can be measured between the centers of the lines, or if the identification features are a series of dots, a distance can be measured between the centers of the dots. The distance of separation is not particularly limited but smaller separation distances are preferred so that the identification is invisible to the unaided eye. For example, the one or more identification features can be separated from each other by an average distance of about 500 microns or less, or more particularly, about 100 microns or less, or more particularly, about 10 microns or less, or more particularly, about one micron or less, or more particularly, about 500 nm or less. Alternatively, the distance can be one micron ore more, such as 3 microns or more.

The stamp can also be characterized by the density of the identification features. For example, a plurality of parallel lines can be generated wherein there is at least one line per every two microns, or at least one line per every one micron, or at least one line per every 0.5 microns or less.

The information associated with the identification region and features may be obtained by determining one or more of the aforementioned characteristics and optionally executing a mathematical function or algorithm.

The surface of the stamp also can comprise one or more marks which can be detected by an optical microscope or an unaided eye. The at least one mark can be located outside of the identification region. Examples include an X shaped mark. The marks, for example, can have lateral dimensions such as line width at the micron scale such as 10 microns or more, at least 50 microns or more, at least 100 microns or more.

The stamp can also comprise the identification regions described above for the pharmaceutical composition. The identification region can be characterized by an identification region area which has an enclosing perimeter around the identification features so that all of the identification features can be found within the enclosing perimeter. This area can be for example, about 10,000 square microns or less, or about 1,000 square microns or less, or about 400 square microns or less, or about 4 square microns or less, or about one square micron or less. The identification region can be, for example a square region with a lateral length and width of 100 microns×100 microns, respectively, or 20 microns×20 microns, or 2 microns×2 microns. Or the identification region can be, for example, a generally rectangular region or circular region. In many cases, two or more identification regions are desired in case one or more of the identification regions become unreadable by scratching, rubbing, or some other undesirable event. For example, the surface of the stamp can comprise more than 20, more than 30, more than 40, or more than 50 identification regions. The identification region can be sufficiently large to be seen by the naked eye or an optical microscope, even when identification features within the identification region can be sufficiently small that they cannot be seen by the naked eye or even with an optical microscope.

The barcode can provide advantages and differences over, for example, a hologram including for example different reading mechanism and optics; providing more information; the size of the larger pattern (not individual line) can be smaller; more diverse geometries and patterns are possible; multiple patterns are possible; and more precise manufacturing conditions can be employed as needed such as for example time, temp, pressure. The identification features can be formed so that they do not form a diffraction grating or effect. In some embodiments, the identification features can form other useful forms such as for example holograms.

The material of the stamp is not particularly limited. In general, the stamp can have a surface which is made of a harder or stiffer material than the material of the surface to be stamped. Materials that can provide high aspect ratio structures can be used. For example, materials can be used which can be subjected to etching processes which result in high aspect ratios such as, for example, reactive ion etching. Stamp material can be, for example, silicon, silicon oxide, quartz, and nickel, and other substrates popular for semiconductor processing.

Stamps can be also treated to increase the durability of the stamp. For example, stamp surfaces can be coated with diamond like coatings (DLC), or nickel films to increase hardness and decrease wear. Stamps fabricated from silicon substrates can be oxidized to increase the hardness of the stamp surface.

A master stamp can be used to produce daughter stamps which are substantially identical but inverted copies of the master.

In another embodiment, indirect nanolithography can be used to selectively remove or modify areas in a resist film coating a substrate. The patterned substrate can be subjected to etching to generate negative relief features, such as trenches, in the substrate. Stamps with negative relief features will result in positive features when used for printing. Negative relief stamps can be replicated to generate secondary stamps that have positive features.

In the nanolithography regime, nanoimprint lithography is a method which can be used in some cases to generate features having lateral dimensions below 200 nm using a stamp on polymer-coated semiconductor wafer. See, for example, U.S. Pat. No. 5,772,905 to Chou (“Nanoimprint Lithography”); U.S. Pat. No. 6,309,580 to Chou; U.S. Pat. No. 6,482,742 to Chou; and U.S. Pat. No. 6,518,189 to Chou. This lithography method can also generate features with lateral dimensions above 200 nm, such as above 600 nm, such as above 1000 nm. The method employs use of stamps having protruding features and made of stiff materials. These references can be used to practice the invention both with respect to the final stamp and the methods of making and using the stamp. Commercial products including stamping instruments and molds or masks are available from Nanonex Corp. (Princeton, N.J.), Suss Microtech AG (Munich, Germany); EV Group (Schareding, Austria); Molecular Imprints Inc (Austin, Tex.); and Obducat (Malmo, Sweden).

Generally, stamp materials which can be used in NIL can be used in this invention. If silicon materials are used, they can be oxidized to silicon dioxide to improve properties such as, for example, durability.

A variety of methods can be used to make the stamp. These methods can be carried out with use of microlithography or nanolithography and can provide excellent high resolution identification features. For example, DPN printing, nanoimprint lithography, microcontact printing, electron beam lithography, ion beam lithography, laser-based lithography, optical lithography, nanografting, and the like can be used.

For example, in one embodiment, the invention provides a method for making a stamp comprising the steps of (i) writing a pattern with a resist material by nanolithography or microlithography on a substrate, (ii) etching the patterned substrate, and optionally (iii) further treating the patterned and etched substrate to form the stamp. For example treatment step (iii) can comprise removing the resist, or any other layers which are undesired in the final stamp.

In one embodiment, direct write nanolithography can be used to pattern a monolayer resist on a substrate. The patterned substrate can be subjected to etching including wet etching or dry etching to remove areas unprotected by the resist. Finally, the resist itself can be removed. The resist can be, for example, a compound which covalently bonds or chemisorbs to the substrate. The resist can be, for example, an alkanethiol on a gold substrate. The substrate can be, for example, fused silica having an outer layer of metal with an intermediate adhesion layer as needed. The direct write nanolithographic method can be carried out with use of a nanoscopic tip to transfer resist material to the substrate by deposition.

In another embodiment, direct write nanolithography can be used to pattern a resist on a substrate. The resist can be a curable material such as, for example, a UV curable or heat curable polymer. Etching can be carried out and the resist removed.

A stamp made by the methods disclosed above can be replicated into secondary stamps, as many identical stamps may be required for commercial production. Furthermore, the identification features present on the master stamp may be replicated multiple times on a secondary stamp, providing redundancy as discussed above. The master stamp may be replicated e.g. by stamping, molding into a soft material (or other methods known to the art), followed by a hardening or coating step, for example polymeric curing, vacuum physical vapor deposition, electroless plating, electroplating or a combination thereof.

In another embodiment, the stamp is replicated on a thin film or foil which is wrapped around a cylinder, then hardened or coated by a hard material. Alternatively, a cylinder may be rolled over the stamp, duplicating the stamp features on said cylinder. See the related art in Chou et al. JVST B 16(6), 1998. The cylinder is then used as a stamp by rolling it over the desired pharmaceutical composition or object.

In the stamping process, variables such as time, temperature, and pressure or force can be varied. These and other variables can be adapted for the particle material to be stamped. For example, stamping can be carried out at for example about 70° C. to about 150° C., or about 85° C. to about 130° C., or about 90° C. to about 130° C. Stamping can be carried out for a time of for example about 0.1 seconds to about 10 seconds, or about 0.5 seconds to about 5 seconds, or about 0.8 seconds to about 3 seconds. The stamping force can be for example about 0.1 kg to about 10 kg, or about 0.5 kg to about 5 kg, or about 1 kg to about 3.5 kg. Force can be converted to pressure based on the stamp area, e.g., 1 mm×0.6 mm. The stamping pressure can be, for example, about 1 MPa to about 200 MPa, or about 5 MPa to about 100 MPa.

One embodiment comprises stamping the bar code onto a surface which has already been molded as a hologram surface.

### Direct Write Lithography

In one method, direct-write nanolithography is used in the process to prepare the stamp. One method is the use of deposition of inks or patterning compounds from a fine, sharp needle like structure which can be, for example, a scanning probe cantilever with or without a tip. The needle like structure can have the patterning compound at the end and can be used to deposit the ink or patterning compound to a substrate. A preferred method of direct-write nanolithography is DPN printing. This method provides for exceptionally high resolution and good patterning capability.

For example, DPN printing technology and etching procedures are described in pending patent application to Mirkin et al. “Fabrication of Solid-State Nanostructures including sub-50 nm Solid-State Nanostructures Based on Nanolithography and Chemical Etching” filed Dec. 3, 2003 (Ser. No. 10/725,939), which is hereby incorporated by reference in its entirety. This application also describes a series of geometric patterns which can be used for the identification features.

In addition, DPN™ printing and deposition methods are extensively described in the following patent applications and patent publications, which are hereby incorporated by reference in their entirety and support the disclosure for the present inventions, particularly with respect to the experimental parameters for carrying out the deposition:

1. U.S. Provisional application 60/115,133 filed Jan. 7, 1999 (“Dip Pen Nanolithography”). This describes applications of deposited monolayers as etch resists.

2. U.S. Provisional application 60/157,633 filed Oct. 4, 1999 (“Methods Utilizing Scanning Probe Microscope Tips and Products Therefor or Produced Thereby”).

3. U.S. Regular patent application Ser. No. 09/477,997 filed Jan. 5, 2000 (“Methods Utilizing Scanning Probe Microscope Tips and Products Therefor or Produced Thereby”), now U.S. Pat. No. 6,635,311 to Mirkin et al. issued Oct. 21, 2003. A wide variety of inks and substrates are described which show chemisorption between the ink and the substrate and can be used as an etch resist.

4. U.S. Provisional application 60/207,713 filed May 26, 2000 (“Methods Utilizing Scanning Probe Microscope Tips and Products Therefor or Produced Thereby”). This application, for example, describes wet chemical etching, working examples, references, and figures, which are all incorporated by reference in their entirety.

5. U.S. Provisional application 60/207,711 filed May 26, 2000 (“Methods Utilizing Scanning Probe Microscope Tips and Products Therefor or Produced Thereby”).

6. U.S. Regular application Ser. No. 09/866,533 filed May 24, 2001 (“Methods Utilizing Scanning Probe Microscope Tips and Products Therefor or Produced Thereby”). This application, for example, describes wet chemical etching, working examples (e.g., example 5), references, and figures, which are all incorporated by reference in their entirety. Computer control of the nanolithographic deposition is also described.

7. U.S. patent publication number 2002/0063212 A1 published May 30, 2002 (“Methods Utilizing Scanning Probe Microscope Tips and Products Therefor or Produced Thereby”).

8. U.S. patent publication number 2002/0122873 A1 published Sep. 5, 2002 (“Nanolithography Methods and Products Produced Therefor and Produced Thereby”).

9. PCT publication number WO 00/41213 A1 published Jul. 13, 2000 based on PCT application no. PCT/US00/00319 filed Jan. 7, 2000 (“Methods Utilizing Scanning Probe Microscope Tips and Products Therefor or Produced Thereby”).

10. PCT publication number WO 01/91855 A1 published Dec. 6, 2001 based on PCT application no. PCT/US01/17067 filed May 25, 2001 (“Methods Utilizing Scanning Probe Microscope Tips and Products Therefor or Produced Thereby”).

11. U.S. Provisional application 60/326,767 filed Oct. 2, 2001, (“Protein Arrays with Nanoscopic Features Generated by Dip-Pen Nanolithography”), now published 2003/0068446 on Apr. 10, 2003 to Mirkin et al.

12. U.S. Provisional application 60/337,598 filed Nov. 30, 2001, (“Patterning of Nucleic Acids by Dip-Pen Nanolithography”) and U.S. regular application Ser. No. 10/307,515 filed Dec. 2, 2002 to Mirkin et al.

13. U.S. Provisional application 60/341,614 filed Dec. 17, 2001, (“Patterning of Solid State Features by Dip-Pen Nanolithography”), now published 2003/0162004 Aug. 28, 2003 to Mirkin et al.

14. U.S. Provisional application 60/367,514 filed Mar. 27, 2002, and publication no. 2003/0185967 on Oct. 2, 2003 to Eby et al. This patent application describes computer control of nanolithographic procedures.

15. U.S. Provisional application 60/379,755 filed May 14, 2002, (“Nanolithographic Calibration Methods”) and U.S. regular application Ser. No. 10/375,060 filed Feb. 28, 2003 to Cruchon-Dupeyrat et al. This patent application describes computer control of nanolithographic calibration procedures.

16. U.S. patent application Ser. No. 10/689,547 filed Oct. 21, 2003 to Crocker et al. (“Nanometer-Scale Engineered Structures, Methods, and Apparatus for Fabrication Thereof, and Application to Mask Repair, Enhancement and Fabrication”). This describes for example use of nanolithography to make photomasks and nanoimprint lithography stamps.

17. U.S. patent application Ser. No. 10/705,776 filed Nov. 12, 2003 to Cruchon-Dupeyrat (“Methods and Apparatus for Ink Delivery to Nanolithographic Probe Systems”). This describes, for example, use of reactive ion etching to make deep structures.

18. U.S. Provisional application 60/544,260 filed Feb. 13, 2004 (“Direct-Write Nanolithography with Stamp Tip: Fabrication and Applications”). This describes, for example, elastomer modification of tips.

19. U.S. Provisional application 60/547,091 filed Feb. 25, 2004 (“Methods for Patterning Conductive Material.”). This describes, for example, use of tipless cantilevers.

In general, state of the art DPN™ printing and deposition-related products, including hardware, software, and instrumentation are also available from NanoInk, Inc. (Chicago, Ill.), and these can be used to carry out the present invention. For example, commercially available products include NSCRIPTOR™, DPN-System-1, environmental chamber, probes, pens, inkwells, substrates, substrate holders, and various accessories including ink dispensing kits, ink dispersion syringes, replacement needles, and probe clips. NSCRIPTOR features for example InkCAD system control, closed loop scanning, and a series of computer programs to facilitate automation. Calibration can be carried out with InkCal. Probes can be single probes, passive multiple probe arrays, active probes, or probes for AC mode.

Parallel methods of the DPN printing process in active mode can be carried out as described in, for example, U.S. Pat. No. 6,642,129 to Liu et al. issued Nov. 4, 2003.

In addition, the following papers describes wet chemical etching procedures used in conjunction with direct-write nanolithography, and is hereby incorporated by reference in its entirety including figures, references, and working examples: Zhang et al., “Dip-Pen Nanolithography-Based Methodology for Preparing Arrays of Nanostructures Functionalized with Oligonucleotides”; Adv. Mat., 2002, 14, No. 20, October 16, pages 1472-1474; Zhang et al., “Biofunctionalized Nanoarrays of Inorganic Structures Prepared by Dip-Pen Nanolithography”; Nanotechnology, 2003, 14, 1113-1117.

The invention provides the use of lithography, including microlithography and nanolithography, in the identification of objects and compositions which are subject to counterfeiting including pharmaceutical compositions. The lithography can be indirectly used to prepare stamps, and then the stamps can be used to provide the identification features on the objects and compositions. Alternatively, the lithography can be used to directly write the identification features on the objects and compositions. Methods which comprise use of deposition of material from a scanning probe microscopic tip can be used including AFM methods. Methods involving cantilevers can be used including both tip and tipless cantilevers.

### Layers of Protection

The process of stamping can provide at least three layers of anti-counterfeiting protection including a shiny spot, a semicovert region, and a covert region. The use of a coating can provide at least two additional layers of protection for use as needed including special reflection or fluorescent properties and chemical properties useful with a detector or upon dissolution.

### NON-LIMITING WORKING EXAMPLES

The various claimed embodiments are further illustrated with use of the following non-limiting working examples.

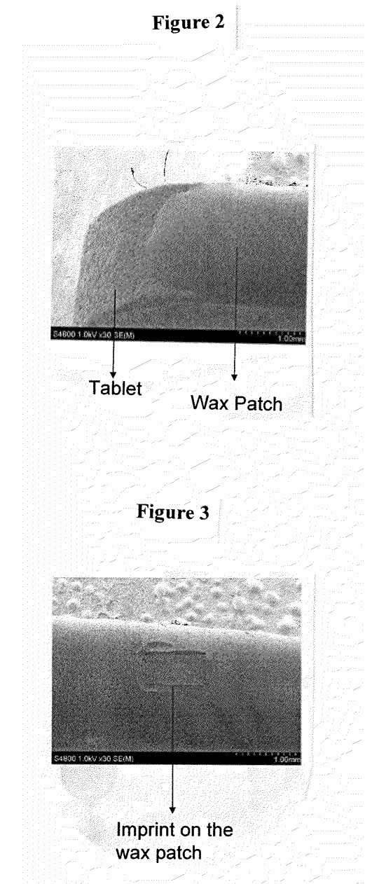

FIG. 2 provides an SEM image of a wax patch on a commercial pharmaceutical tablet.

FIG. 3 provides an SEM image of an imprint on the wax patch of FIG. 2.

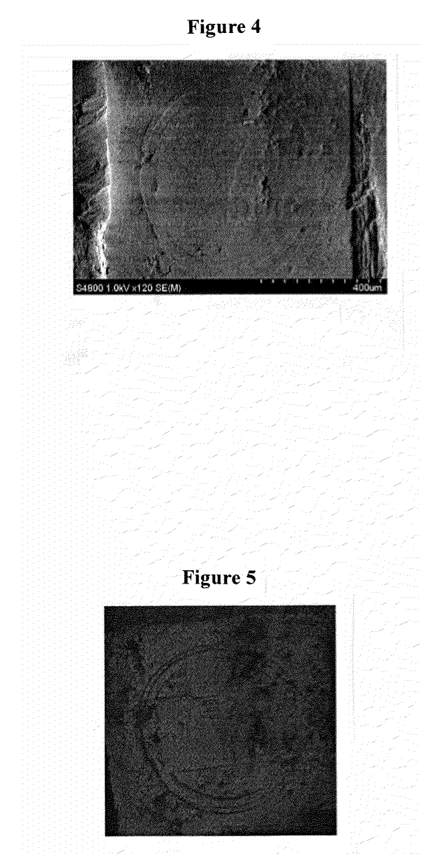

FIG. 4 provides an optical image of a logo on a wax patch on a commercial pharmaceutical tablet.

FIG. 5 provides an SEM image of a logo on a wax patch on a commercial pharmaceutical tablet.

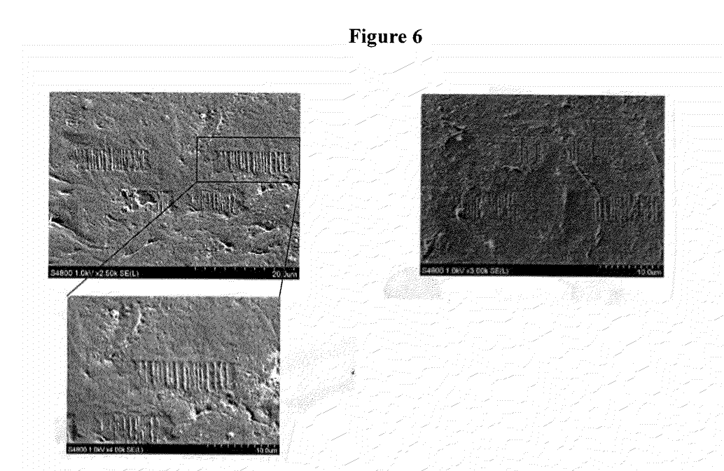

FIG. 6 provides SEM images of nanofeatures and barcodes created on a wax patch.

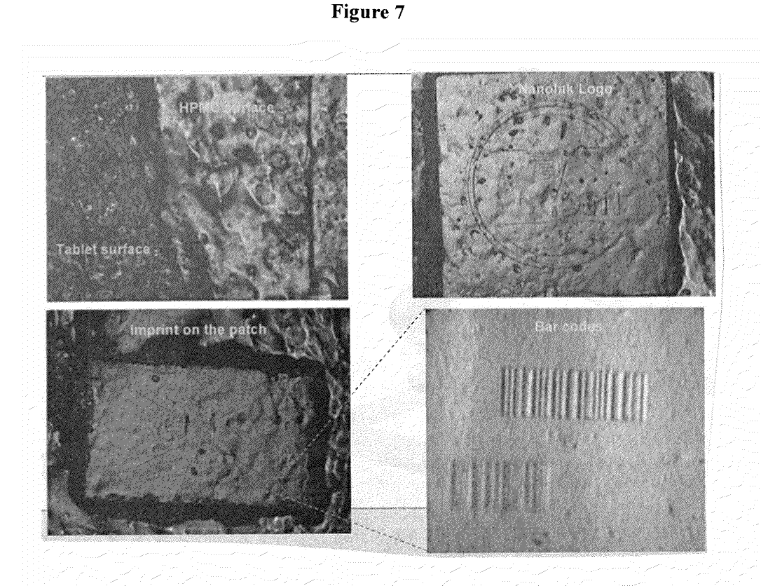

FIG. 7 provides optical images for HPMC patch on a commercial pharmaceutical tablet.

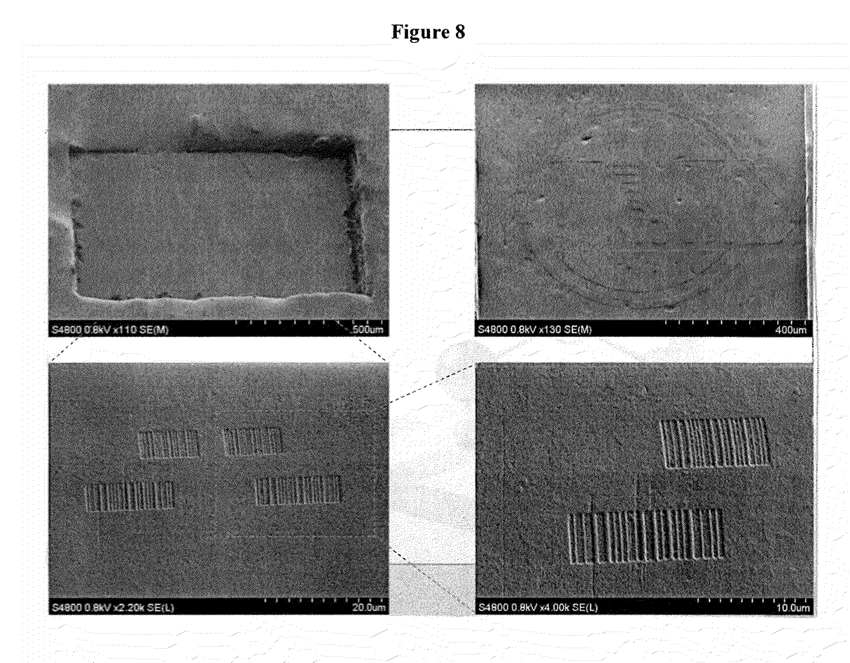

FIG. 8 provides SEM images for HPMC patch on a commercial pharmaceutical tablet.

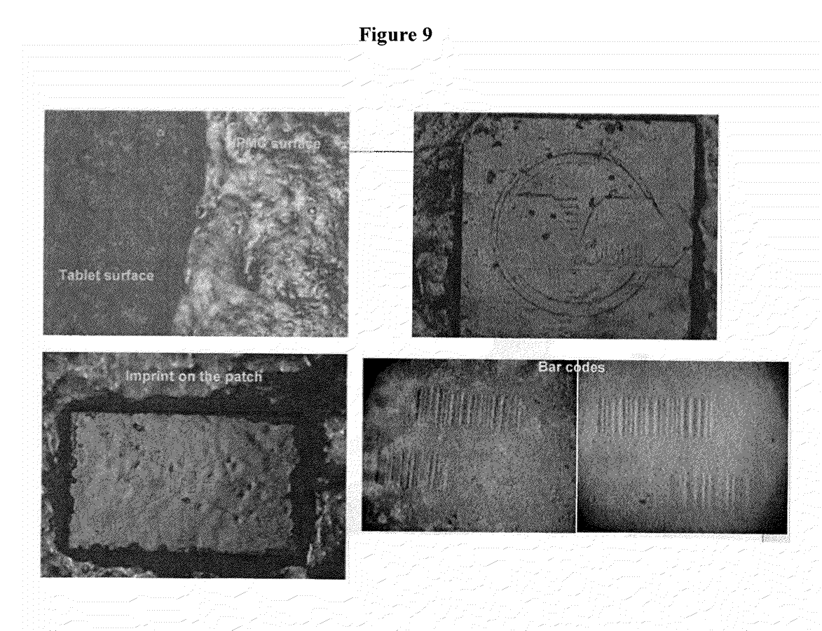

FIG. 9 provides more optical images for HPMC patch on a commercial pharmaceutical tablet.

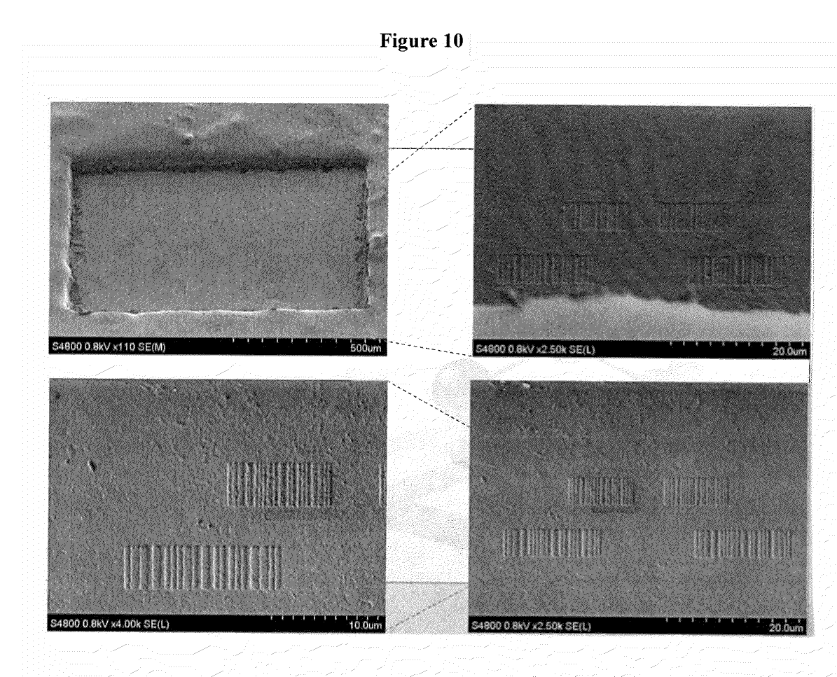

FIG. 10 provides more SEM images for HPMC patch on a commercial pharmaceutical tablet.

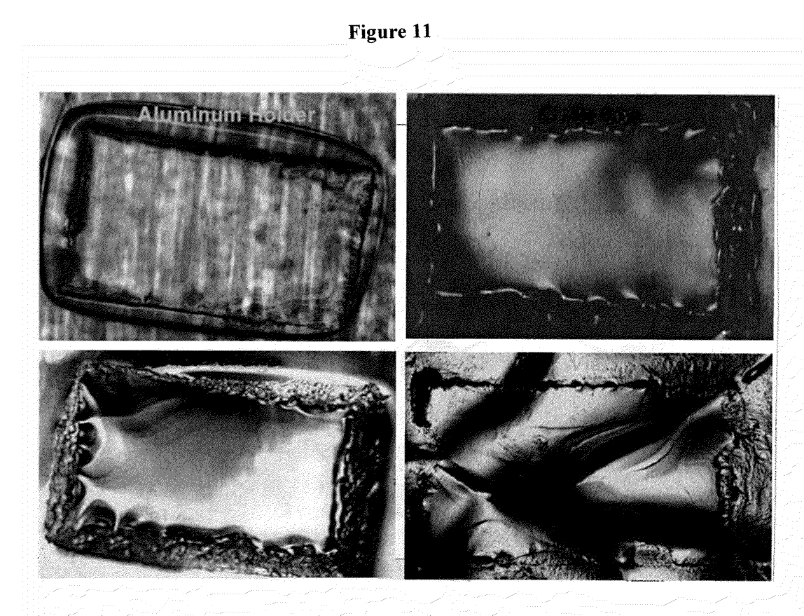

FIG. 11 provides optical images for an epoxy patch on different surfaces.

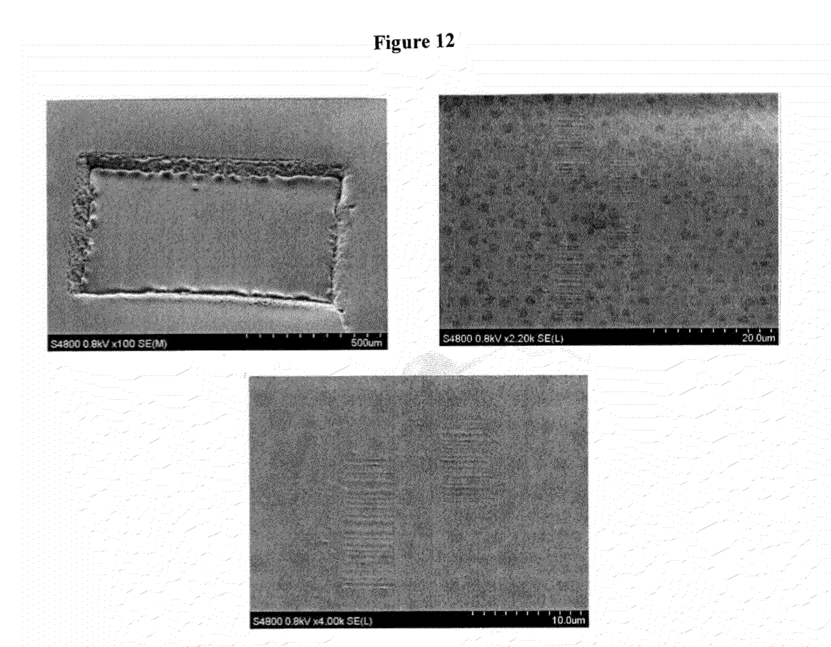

FIG. 12 provides SEM images for an epoxy patch on a commercial pharmaceutical box.

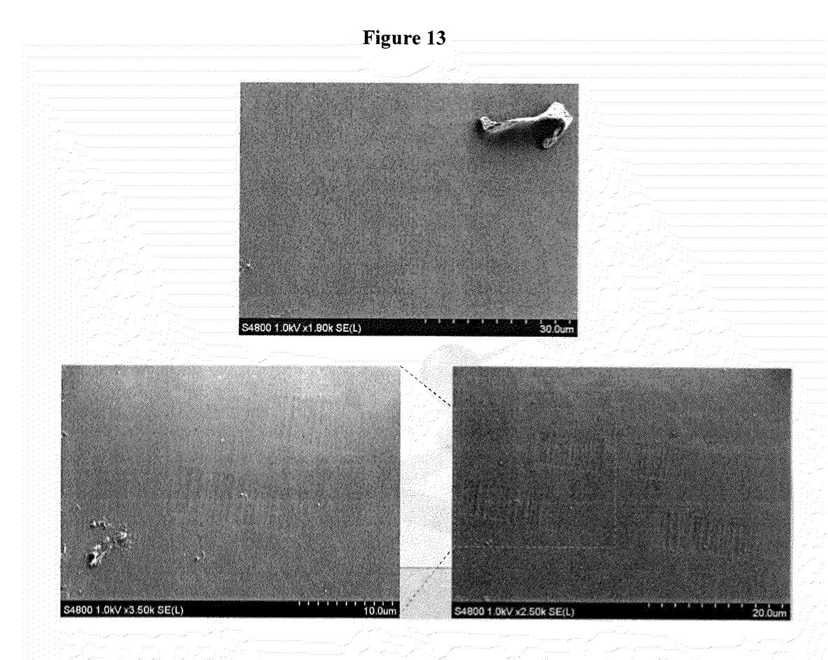

FIG. 13 provides SEM images for an epoxy patch on a commercial pharmaceutical box.

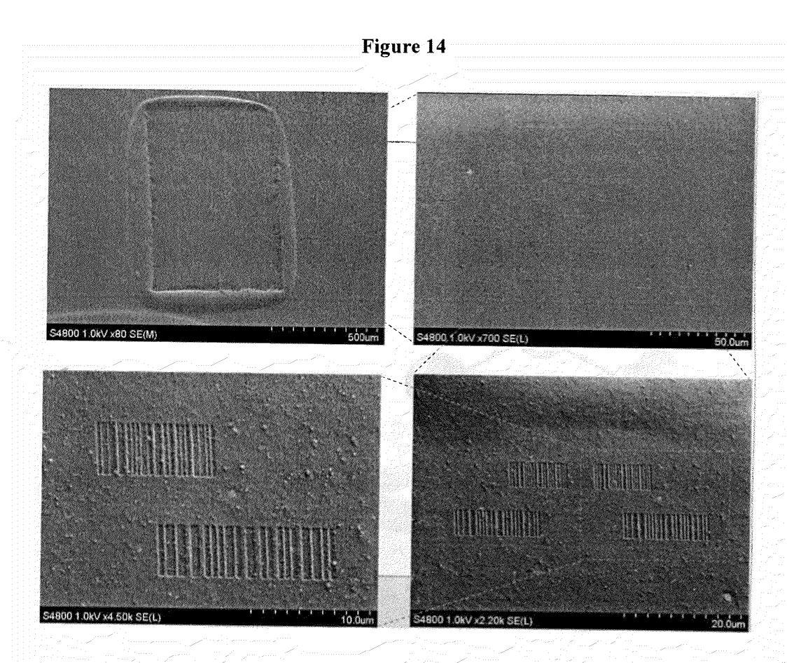

FIG. 14 provides SEM images for an epoxy patch on an aluminum holder.

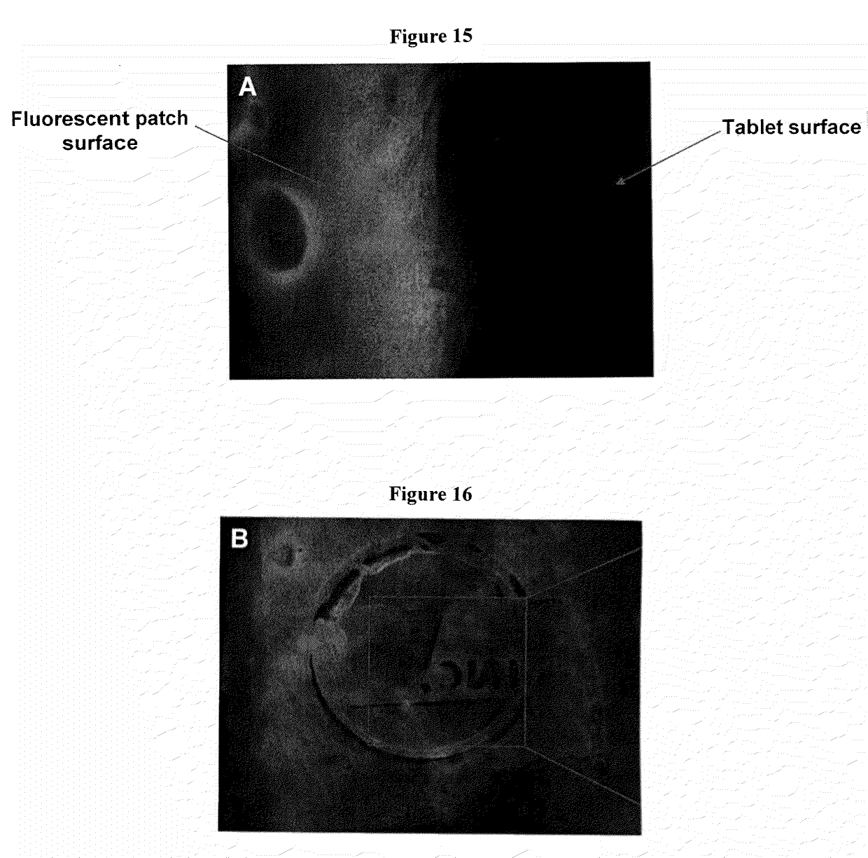

FIG. 15 illustrates use of a fluorescent dye and a fluorescent microscope.

FIG. 16 illustrates identification logo found with use of a fluorescent dye and a fluorescent microscope.

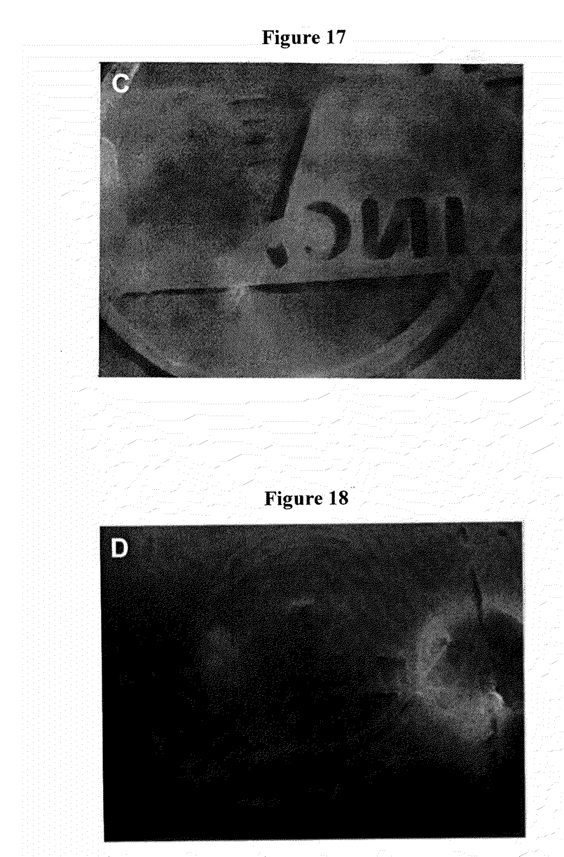

FIG. 17 illustrates identification logo found with use of a fluorescent dye and a fluorescent microscope at higher magnification.

FIG. 18 illustrates coating an imprinted area with a transparent, non-fluorescent coating and a logo can be still seen with a fluorescent microscope.

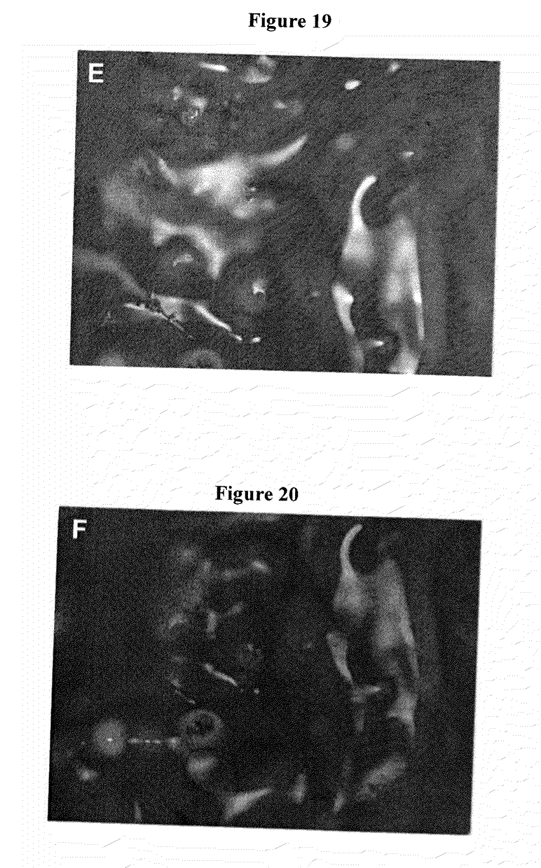

FIG. 19 illustrates same area as FIG. 18 but with use of an optical microscope.

FIG. 20 illustrates same area as FIG. 18 but with use of an optical microscope

### Example 1

### Fluorescent Embodiment

A fluorescent dye was included in the coating. In particular, the use of one or more fluorescent dyes, including intercalating dyes, mixed or tagged with the patch material or a chemical tag can be used to detect the chemical properties of these entities upon dissolution of the pharmaceutical units. In addition, the fluorescent patch can provide an additional layer of protection similar to the shiny spot protection layer upon shining an appropriate UV light (or using a routine UV transilluminator) as seen in FIG. 15. In this image a fluorescent patch applied to a tablet surface is observed under a fluorescent microscope and only the fluorescent patch can be seen. FIGS. 16 and 17 are fluorescent images of a logo imprinted on the fluorescent patch.

An additional layer of protection can be achieved if the imprinted area is masked with a transparent non-fluorescent polymer, the masking polymer can provide a protection to the imprint from physical damages and the covert or overt imprint can be seen only by a fluorescent microscope. As seen in FIG. 18, the imprinted area was masked with an epoxy “Krazy Glue”, after masking the total patch area, the NanoInk logo can still be seen using the fluorescent microscope. The same area under a regular optical microscope is seen as a rough surface of the deposited epoxy, FIGS. 19 and 20. In some instance the surface of the epoxy is cracked upon drying and the imprint can not be seen under fluorescent unless a fresh solution of epoxy is applied onto the imprinted area to fill up these cracks to form a homogeneous layer.

The formulation was made comprising HPMC and a solvent, water, and the fluorescent dye, Fluorescein. Several microliters of formulation were smeared onto the surface of a commercial tablet with use of an Eppendorf pipette and tips. The coating was cured in an oven at a temperature of about 45-60 C for 2-6 minutes. The coating was stamped with a nickel stamp for one second, at 125 C, and a pressure of 2.5 kg. The coating was further overcoated with an all purpose Instant Krazy Glue from Elmers Product Inc. Fluorescent and optical images were obtained with use of a Zeiss Axioplan optical and fluorescent microscope using a 5× or 10× objective lens.

Dyes are known in the art and fluorescent dyes are a category of dyes for which the functional concepts and actual structures are known in the art. See for example U.S. Pat. Nos. 6,630,437, 6,955,872, and 6,972,339. General classes of dyes include for example stilbeness; coumarin and carbostyril compounds; 1,3-diphenyl-2-pyrazolines; naphthalimides; benzazdyl substitution products of ethylene, phenylethylene, stilbene, thiophene; and combined hateroaromatics.

### Example 2

### Fabrication Process of a PDMS Patch

In this embodiment, poly(dimethylsiloxane) (PDMS) was used as the patch coating material on the coated tablet. The PDMS used was based on Sylgard 184 Silicone Elastomer obtained from Dow Corning (Mich., USA).

A curing (or crosslinking) agent and the base silicone elastomer material were thoroughly mixed in a 1:10 weight ratio. The pre-polymer mixture was degassed in ambient for about 1 hour to remove any air bubbles in the mixture and ensure complete mixing of the two parts. The pre-polymer mixture was then applied onto a pharmaceutical 1 mg tablet surface and cured at ambient temperature for about 12 hours.

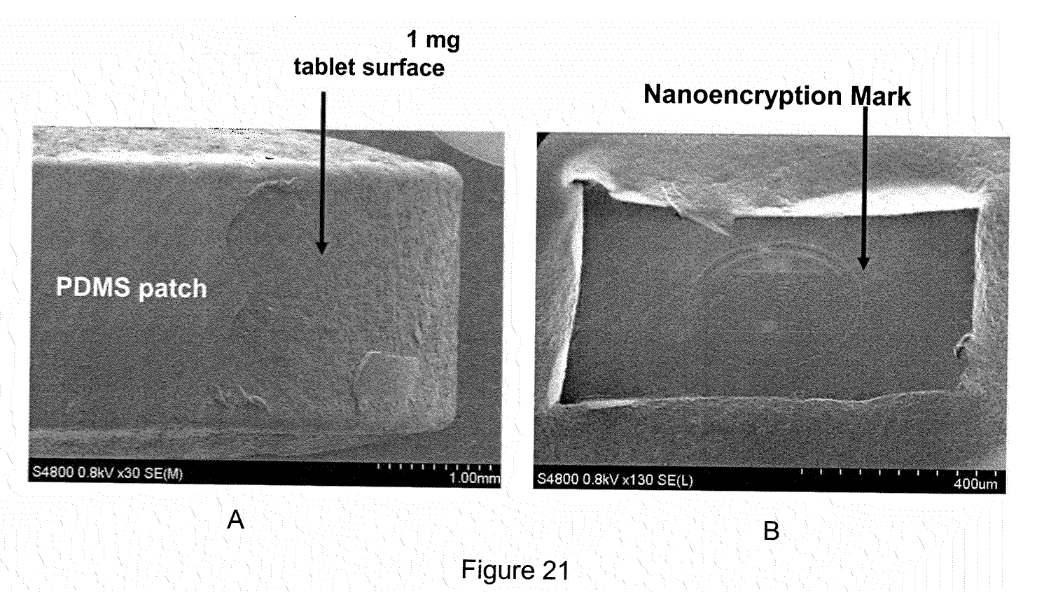

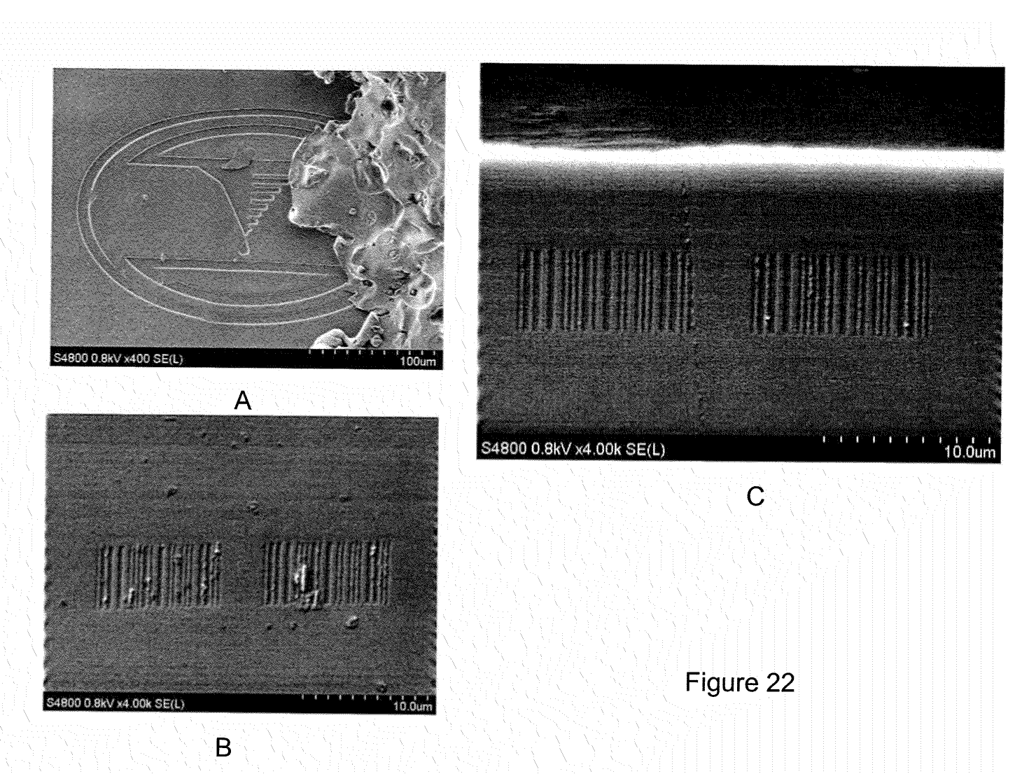

FIGS. 21A-21B provide SEM images of a PDMS patch and a Nanoencryption™ mark on the patch, respectively. FIGS. 22A-22C provide SEM images of micro- and nano-scale features on PDMS patch on a pharmaceutical 1 mg tablet.

FIGS. 21 and 22 demonstrate that the PDMS patch can be easily applied to the coated tablets that in turn can be used as the surface for Nanoencryption. Also, using PDMS as the patch material allowed features at both micro and nano scales to be fabricated on coated tablets. Thus, PDMS can serve as a desirable patch coating material to enable Nanoencryption on many materials and surface.

1. A method for imprinting pharmaceutical unit compositions comprising:
providing a pharmaceutical unit composition,
partially coating the exterior of the composition with a coating,
stamping the coating with a stamp comprising a plurality of identification features, wherein identification features from the stamp are at least partially transposed in the coating and form a barcode in the coating,
wherein the plurality of identification features comprise at least one lateral dimension of about 1,000 nm or less.

2-3. (canceled)

4. The method according to claim 1, wherein the coating comprises a composition which is generally recognized as safe (GRAS).

5-11. (canceled)

12. The method according to claim 1, wherein the coating is applied to only a portion of the exterior of the pharmaceutical unit composition.

13-21. (canceled)

22. The method according to claim 1, wherein the plurality of identification features comprise at least one lateral dimension of about 500 nm or less.

23. (canceled)

24. The method according to claim 1, wherein the coating comprises at least one fluorescent dye.

25-26. (canceled)

27. The method according to claim 1, wherein the identification features form both overt and covert marks.

28-29. (canceled)

30. The method according to claim 1, wherein the coating comprises a polymer, and wherein the plurality of identification features comprise at least one lateral dimension of about 500 nm or less, and wherein stamping is carried out at a temperature of about 70° C. to about 150° C., and wherein stamping is carried out at a pressure of about 1 MPa to about 200 Mpa, and wherein stamping is carried out for a time of about 0.1 second to about 10 seconds.

31. A method for imprinting objects comprising:
providing an object,
partially coating the exterior of the object,
stamping the coating with a stamp comprising a plurality of identification features, wherein identification features from the stamp are at least partially transposed in the coating to form a barcode,
wherein the plurality of identification features comprise at least one lateral dimension of about 1,000 nm or less.

32. The method according to claim 31, wherein the object comprises a pharmaceutical composition.

33. (canceled)

34. The method according to claim 31, wherein the coating provides a smoother surface than the object exterior which is coated.

35-36. (canceled)

37. The method according to claim 31, wherein the coating comprises at least one fluorescent dye.

38-40. (canceled)

41. A method for imprinting pharmaceutical unit compositions comprising:
providing a pharmaceutical unit composition,
partially coating the exterior of the composition with a coating which is adapted to receive a barcode stamp and also bind to the pharmaceutical unit composition,
stamping the coating with a stamp comprising a plurality of identification features, wherein temperature, pressure, and time are adapted so that identification features from the stamp are at least partially transposed in the coating and form a barcode in the coating,
wherein the plurality of identification features comprise at least one lateral dimension of about 1,000 nm or less.

42-47. (canceled)

48. An imprinted pharmaceutical unit composition comprising:
pharmaceutical unit composition,
a coating disposed on the exterior of the composition,
a plurality of identification features in the coating and forming a barcode in the coating,
wherein the plurality of identification features comprise at least one lateral dimension of about 1,000 nm or less.

49. An imprinted object comprising:
an object,
a coating disposed on the exterior of the object,
a plurality of identification features in the coating and forming a barcode in the coating,
wherein the plurality of identification features comprise at least one lateral dimension of about 1,000 nm or less.

50. (canceled)

51. A method for imprinting pharmaceutical unit compositions comprising:
providing a pharmaceutical unit composition,
partially coating the exterior of the composition with a coating,
stamping the coating with a stamp comprising a plurality of identification features, wherein identification features from the stamp are at least partially transposed in the coating and form a barcode in the coating,
wherein the plurality of identification features comprise at least one lateral dimension of at least about 1,000 nm.

52-53. (canceled)

55. The method according to claim 51, wherein the coating comprises more than one type of coating material.

56-57. (canceled)

58. The method according to claim 51, wherein the coating comprises a silicone elastomer.

59. The method according to claim 51, wherein the coating comprises polydimethylsiloxane.

60-70. (canceled)

71. The method according to claim 1, wherein the coating comprises an elastomeric polymer.

72. The method according to claim 1, wherein the coating comprises an elastomeric polymer having a glass transition temperature of less than about −50° C.

73. The method according to claim 1, wherein the coating comprises a siloxane polymer.

74. The method according to claim 1, wherein the pharmaceutical unit composition comprises an uncoated tablet.
